(19) United States
(12) Patent Application Publication
(10) Pub. No.: US 2026/0317790 A1
(43) Pub. Date: Oct. 15, 2026

(54) Process for Hydrogen Production from Water by Photosynthesis

(71) Applicant: Helmholtz-Zentrum für Umweltforschung GmbH-UFZ, Leipzig (DE)

(72) Inventors: Andreas SCHMID, Leipzig (DE); Samuel GRIMM, Leipzig (DE); Christian DUSNY, Leipzig (DE); Sebastian ROTHER, Leipzig (DE); Ron STAUDER, Halle (DE); Stephan KLÄHN, Leipzig (DE)

(21) Appl. No.: 19/475,302

(22) PCT Filed: Apr. 28, 2023
(86) PCT No.: PCT/EP2023/061376

§ 371 Date: Oct. 14, 2025

(51) Int. Cl. C12P 3/00; B01D 53/22; B01D 71/40; B01D 71/70; C12M 1/00; C12M 1/04; C12M 3/06; C12P 39/00

(52) U.S. Cl. CPC C12P 3/00; B01D 53/228; B01D 53/229; B01D 71/401; B01D 71/701; C12M 21/02; C12M 23/16; C12M 23/22; C12M 23/24; C12P 39/00; B01D 2256/16; B01D 2257/104

(57) ABSTRACT

Methods are disclosed for production of hydrogen from water with sunlight as energy source and oxygen sensitive hydrogenases catalyzing the formation of hydrogen in the light. Photobioreactor for performing the methods are also disclosed.

### FIELD OF THE DISCLOSURE

The present invention relates to a method for the production of hydrogen from water with sunlight as energy source and oxygen sensitive hydrogenases catalyzing the formation of hydrogen in the light and photobioreactor for performing the method.

### TECHNOLOGICAL BACKGROUND

Recent reports of the Intergovernmental Panel on Climate Change (IPCC) on global warming, usage and exploitation of land and marine resources demonstrate once more the need to implement and extent sustainable and climate neutral product cycles. Since the use of fossil fuels largely contribute to CO2 emission, new alternatives for renewable resources and energy carrier need to be discussed and developed.

Hydrogen is a key component for transforming economies and societies towards a CO₂ neutral generation of energy carriers and the use of energy and materials in a sustainable way. Hydrogen is the energy carrier of the future, as on the one hand it has a high combustion energy and on the other hand represents an environmentally friendly energy source. It is available in huge quantities and can be obtained from water using various methods. Hydrogen can be converted back into current and heat in a fuel cell, with the end product in turn being just water. At least theoretically, enormous quantities of energy can also be buffered in the long term by means of hydrogen.

Photocatalytic water splitting, meaning splitting water into hydrogen and oxygen by means of sunlight, represents a technologically important alternative for energy-expensive electrolytic water splitting. In this case, generally, algae or cyanobacteria are used which, under certain conditions, can give off hydrogen to the environment during the metabolic process. In this way, sunlight and water can be converted directly into hydrogen by means of photosynthesis in bio-reactors with eucaryotic algae or cyanobacteria. Thus, the formation of photo-hydrogen from cyanobacteria by photosynthesis has a significant technical potential for providing this energy carrier.

Phototrophic microorganisms have the potential to function as biocatalysts made of carbon dioxide (CO₂), water, and salts for the synthesis of high energy compound by mainly using light for water splitting. By genetic engineering and optimization of growth conditions, the cyanobacterium Synechocystis sp. PCC 6803 was optimized as a model organism to produce a variety of valuable and energy-rich carbon compounds as well as H₂ as an energy carrier of the future. H₂ is produced by transferring electrons from the photosystem or metabolites to hydrogen via hydrogenases (see FIG. 1). All hydrogenases are inactivated to a larger or smaller extent by oxygen, limiting hydrogen production by oxygenic phototrophic microorganisms. The native hydrogenase of Synechocystis sp. PCC 6803 is a representative of highly oxygen-sensitive hydrogenases. Anaerobic reaction environments are necessary for keeping the enzyme active. To obtain anaerobic conditions, expensive oxygen scavengers such as reduced intracellular storage compounds, extracellular compounds used for intracellular oxygen depletion, metals or enzymes have been used, but their use in large-scale application is limited or economically unfavorable. New reactor designs are necessary to actively remove excess oxygen and products, control growth and efficiently supply microbes with substrates (light and CO₂). Today, physiological concepts (inhibitors and stress conditions) and molecular concepts (e.g. gene fusion of hydrogenases and photosystem I) can be applied to produce photohydrogen.

Due to the oxygen sensitivity of hydrogenases, an efficient oxygen removal is necessary to enable a prolonged and continuous hydrogen production via oxygenic photosynthesis. Several methods to deplete oxygen from the cultivation medium and the intracellular reaction environment of hydrogenases are described for stabilizing these in living hydrogen forming microbial cells. They comprise reactive reduction of oxygen by chemical and/or biological means combined with cellular respiration and metabolism or temporal separation of anaerobic and aerobic (active with oxygenic photosynthesis) cell cultures. To date, none of these methods is sufficient for developing a process concept to produce in a continuous way hydrogen from water with natural photosynthesis in a process window matching economical needs.

Therefore, the object of the invention is to provide a method for producing hydrogen from water with natural photosynthesis and during active oxygenic photosynthesis in a continuous and prolonged way which separates undesirably produced molecular oxygen from the chemical production of a target product and prevents the disadvantages of the prior art, such as the risk of explosion due to the formation of oxyhydrogen gas, two process phases and inactivation of oxygen-sensitive enzymes.

### SUMMARY OF INVENTION

The invention is defined by the appended claims.

The invention pertains to a method for producing hydrogen from water comprising the following steps of co-cultivation of hydrogen(H₂)-releasing cyanobacteria with heterotrophic, oxygen respiring bacteria and an organic electron source under anaerobic conditions in a reaction medium containing water and/or a biofilm and exposure of the co-culture to light to induce photohydrogen evolution, wherein the oxygen is exclusive extracellular removed with a membrane area-specific withdrawal rate high enough to deplete oxygen from the cytoplasm of the hydrogen forming phototrophic microorganism, preferably at a rate of 0.5 to 9 μmolO₂*mm²*h⁻¹ to an oxygen concentration of 0 to 12 % v/v. This rate greatly exceeds the net rate of oxygen being produced by photosynthesis in the here described photobioreactor concept thus allowing efficient removal of oxygen away from the cellular environment.

The present invention is based on the effect that exclusive extracellular depletion of oxygen is sufficient to keep oxygen labile hydrogenases active in a cell actively performing oxygenic photosynthesis and not depleting intracellular oxygen by cellular respiration to a significant extent, i.e., net oxygen production is close or equal to gross oxygen from photosynthesis. Furthermore, the process according to the invention for the production of hydrogen from water uses electrons derived exclusively from the microbial electron transport chain in the cellular membranes during running oxygen producing photosynthesis in the same microbial cell. The majority of electrons for the formation of hydrogen should be delivered directly by water and not via carbon metabolism in the cell. This allows hydrogen production with a theoretical possibility of a high energy conversion efficiency with electrons generated from water being directly transferred via the photosynthetic electron transport chain towards hydrogenase, without cellular carbon metabolism playing a major role es electron source in electron flux. Therefore, with the method according to the invention it is possible to keep the intracellular oxygen labile hydrogenases active over two to three weeks.

In a further aspect, the invention provides a photobioreactor to perform the method according to the invention, comprising a chip made of a transparent, biocompatible, and gas-tight polymer, capillary members which are arranged meander-shaped, parallel on the chip, wherein at least one capillary member is the reaction channel flanked by at least one capillary member which is the gas channel and between the reaction channel and the gas channel are integrated oxygen-and hydrogen-selective polymer membranes to remove hydrogen and oxygen from the reaction channel. The photobioreactor according to the present invention provides the possibility to remove the oxygen with high efficiency in combination with the high area-specific productivity of microreactors.

It has been shown that exclusive extracellular degradation of oxygen is sufficient to keep oxygen labile hydrogenases active in a cell actively performing oxygenic photosynthesis.

### BRIEF DESCRIPTION OF THE DRAWINGS

Features will become apparent to those of ordinary skill in the art by describing in detail exemplary embodiments with reference to the attached drawings in which:

FIG. 1 illustrates the values for H₂, O₂, and CO₂ exchange in the Synechocystis PCC 6803 ndhB mutant M55 in darkness and during a 25-min illumination period based on mass spectrometric measurements.

FIG. 2 illustrates the microchannel concept and geometry of the photobioreactor.

FIG. 3 shows the H₂ production in mixed species biofilm capillaries ( M55/Pseudomonas).

### DETAILED DESCRIPTION OF THE INVENTION

The present disclosure may be embodied in various different forms and should not be construed as being limited to only the illustrated embodiments herein. Rather, these embodiments are provided as examples so that this disclosure will be thorough and complete, and will fully convey the aspects and features of the present disclosure to those skilled in the art.

As used herein, the term “and/or” includes any and all combinations of one or more of the associated listed items. Further, the use of “may” when describing embodiments of the present disclosure refers to “one or more embodiments of the present disclosure.” In the following description of embodiments of the present disclosure, the terms of a singular form may include plural forms unless the context clearly indicates otherwise.

As used herein, the term “substantially,” “about,” and similar terms are used as terms of approximation and not as terms of degree, and are intended to account for the inherent deviations in measured or calculated values that would be recognized by those of ordinary skill in the art. Further, if the term “substantially” is used in combination with a feature that could be expressed using a numeric value, the term “substantially” denotes a range of +/−5% of the value centered on the value.

It will be further understood that the terms “include,” “comprise,” “including,” or “comprising” specify a property, a region, a fixed number, a step, a process, an element, a component, and a combination thereof but do not exclude other properties, regions, fixed numbers, steps, processes, elements, components, and combinations thereof.

As used herein, the term “biofilm” refers to a community of microorganisms, e.g. bacteria, in a film-like common matrix of extracellular polymeric material, for example, polysaccharides and/or proteins. It may be a community of microorganisms of the same species or of different species. For example, autotrophic cyanobacteria may be associated with heterotrophic bacteria in a biofilm.

As used herein, the term “anaerobic” means under exclusion of oxygen or at least under strongly oxygen-limited conditions.

Photoautotrophic microorganisms are capable to produce organic compounds (such as carbohydrates, fats, and proteins) from simple, usually inorganic, compounds, thereby using light as energy source. For example, photoautotrophic microorganisms can exploit water as an electron source, use light as an energy source, and fix CO₂ as a carbon source. Photoautotrophic microorganisms are a special case of phototrophic microorganisms.

[symbol] duction of oxygen by photosynthetic water oxidation means particularly the following reaction, carried out by the photoautotrophic microorganisms:

O₂+4 H+4e

Photoautotrophic microorganisms according to the present invention are hydrogen(H₂)-releasing cyanobacteria.

Chemoheterotrophic microorganisms use organic compounds that are already available, and not self-produced by the chemoheterotrophic microorganism, as energy source. Chemoheterotrophic organisms are unable to fix carbon to form their own organic compounds. So, chemoheterotrophic microorganisms use organic compounds both as energy source and as carbon source.

In one embodiment, the photoautotrophic microorganisms are hydrogen-releasing cyanobacteria which are selected from the genus Synechocystis, particularly Synechocystis sp. PCC 6803 (Pasteur Culture Collection accession No. PCC 6803) or any derivative thereof. Basically, all photoautotrophic microorganisms carrying out natural water splitting photosynthesis and all oxygen sensitive hydrogenases can be used for this process. Hydrogenases with a bias towards proton reduction (hydrogen formation) over hydrogen oxidation are preferred. Likewise, phototrophic microorganisms with a metabolic capability of hydrogen release upon cellular overreduction by oxygenic photosynthesis are a preferred.

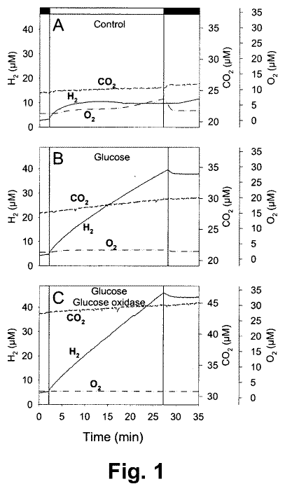

The mutant of Synechocystis PCC6803 named M55 (Ogawa et al., Proc. Natl. Acad. Svi. USA, 1991, 4275 to 4279) is able to form hydrogen in the light for a limited amount of time, if glucose or glucose oxidase and glucose is present in the cultivation medium. As illustrated in FIG. 1 under these conditions, the cellular respiration metabolizing glucose to CO₂ and water with depletion of oxygen is sufficient to reduce the oxygen amount inside the cell far enough to keep the oxygen labile hydrogenase of Synechocystis PCC6803 active for forming hydrogen. In the context of respiration as an intracellular sink for depleting intracellular oxygen, it is important to note, that dark respiration is also repressed in Synechocystis PCC6803 M55.

The M55 mutant of Synechocystis PCC6803 shows a defect in the ndhB gene and hence, has no NDH-1 complexes. Derivatives of Synechococcus PCC6803 may be generated by other genetic engineering as GMO with a hydrogen forming phenotype similar or identical to Synechococcus PCC6803.

In a preferred embodiment the H₂-releasing cyanobacteria are generated by molecular breeding with a hydrogen forming phenotype similar or identical to Synechococcus PCC6803 wild-type or Synechocystis sp. PCC 6803.

In one embodiment, the chemoheterotrophic microorganisms are selected from heterotrophic, oxygen respiring bacteria. In a more specific embodiment the heterotrophic, oxygen respiring bacteria are selected from the genus Pseudomonas, particularly Pseudomonas sp. VLB120, more specifically Pseudomonas taiwanensis VLB120_pCom10_capro (Deutsche Sammlung von Mikroorganismen und Zellkulturen, DSMZ accession No. DSM 32333), or Pseudomonas taiwanensis VLB120AC (Deutsche Sammlung von Mikroorganismen und Zellkulturen, DSMZ accession No. DSM 24711).

Pseudomonas taiwanensis was investigated for a long-term oxygen removal to enable a photosynthesis-driven hydrogen production in the M55 mutant of Synechococcus PCC6803.

The growth of Pseudomonas cells requires external reduced electron sources, e.g. organic compounds. According to the present invention the organic electron source is selected from citrate, gluconate or glycerol. In a preferred embodiment the organic electron source is citrate. Alternative carbon sources which do not provide significant amounts of electrons to the formation of hydrogen can likewise be used.

The co-cultivation comprises Synechocystis sp. PCC 6803 and P. taiwanensis or any derivatives thereof. Thus, the heterotrophic bacterial strain, Pseudomonas taiwanensis VLB120 is used in a dual species cultivation system to actively reduce oxygen (O₂) produced by the cyanobacterium by respiration. Citrate, gluconate or glycerol are not used as carbon source by Synecchocystis sp. PCC 6803. In contrast, glucose is metabolized by Synechocystis sp. PCC 6803 which deregulates photosynthesis and is feeding electrons to the hydrogenase. The method according to the present invention makes sure that the hydrogenase is active during oxygenic photosynthesis even when oxygen (O₂) is only withdrawn from outside of the cyanobacterial cell.

According to the present invention the reaction vessel for the co-cultivation contains water, nutrient salts, microbial cells and/or a biofilm.

The biofilm comprises the hydrogen-releasing cyanobacteria and heterotrophic, oxygen respiring bacteria, preferably Synechocystis sp. PCC 6803 and P. taiwanensis or any derivatives thereof and extracellular polymeric substances (EPS). The hydrogen-releasing cyanobacteria and heterotrophic, oxygen respiring bacteria may be partially or wholly embedded in EPS. EPS are secreted by the hydrogen-releasing cyanobacteria and/or heterotrophic, oxygen respiring bacteria. EPS may comprise one or more of a substance that is selected from polysaccharides, proteins, lipids, and DNA.

The biofilm may be a biofilm which is obtained by cultivation of a mixture of the hydrogen-releasing cyanobacteria and heterotrophic, oxygen respiring bacteria, particularly on a surface. In one embodiment, the thickness of the biofilm is from about 10 pm up to about 500 pm. The thickness of the biofilm may vary.

In one embodiment, the biofilm is adhered to a surface of a carrier. Preferably, the biofilm is produced, or grown, on the carrier. The carrier is preferably an artificial carrier. An artificial carrier is either man-made or based on a natural carrier which is modified by an artificial (i.e. man-made) process. The carrier may be made from numerous substances, wherein particularly suitable substances are glass, ceramic, plastic, metal, or a combination thereof.

The carrier may be made from a translucent, i.e. light-transmissive, material.

In one embodiment, the chip-based carrier has the shape of a tube or capillary and the biofilm is preferably located on an inner surface. The carrier may be a part of the reactor which is described hereinafter.

Light may be any light that is suitable to provide energy for photoautotrophic microorganisms, particularly to induce photosynthesis, even more particularly to induce photosynthetic water oxidation. Light may be visible light, such as daylight. Possible light spectra are given in A. Hoschek et al., Angew Chemie Int Ed 2017; 56:15146-15149, which is incorporated by reference.

The biofilm capillary reactor for conducting the method according to the invention comprises at least one capillary member, at least one reservoir for at least one liquid phase, and at least one reservoir for a gas phase, wherein the at least one reservoir for a liquid phase and the at least one reservoir for a gas phase are in interruptible fluid connection with the at least one capillary member.

The reactor which can be used in the present invention is described in patent application WO 2018/162465 A1 and WO 2020/052762 A1.

In a further embodiment the gas phase above the reaction medium is periodically exchanged by nitrogen (N₂) gas, for example every two days. This leads to an improved hydrogenase activity and an improved stability for the reaction system according to the method according to the present invention.

Due to the use of the biofilm capillary reactor for conducting the method according to the invention it is possible to provide a technical concept for the membrane-assisted physical removal of oxygen.

A further aspect of the present invention is a photobioreactor to perform the method according to the present invention comprising a chip made of a transparent, biocompatible, and gas-tight polymer, capillary members which are arranged meander-shaped, parallel on the chip, wherein at least one capillary member is the reaction channel flanked by at least one capillary member which is the gas channel and between the reaction channel and the gas channel (are integrated oxygen-and hydrogen-selective polymer membranes to remove hydrogen and oxygen from the reaction channel.

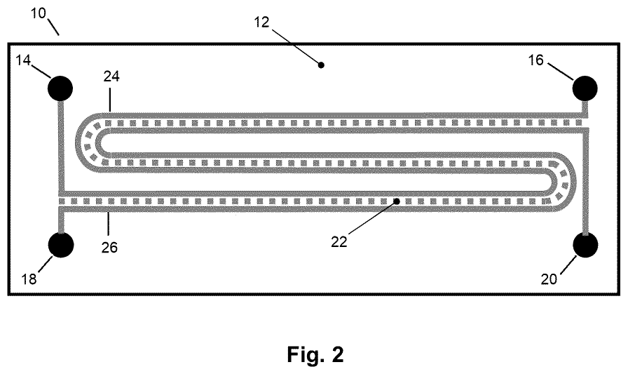

FIG. 2 shows micro-channel geometry and concept of the photobioreactor 10. The micro-molded gas channels comprise a gas channel inlet 14 and gas channel outlet 16 as well as a reaction channel inlet 18 and a reaction channel outlet 20 which connect the gas channel 24 and reaction channel 26 containing the membrane 22. The chip channel network structure is positioned on a glass substrate 12.

In a further embodiment of the photobioreactor 10 the gas-tight polymer is an acrylate or any other polymer with low hydrogen and oxygen permeability that can be used for micro-molding. In a further embodiment of the photobioreactor 10 the microcapillaries 24, 26 have a diameter between 50 μm to 1000 μm, preferably with a diameter below 200 μm and have a length of 85 to 600 cm per chip.

In a further embodiment of the photobioreactor 10 the membranes 22 are made of polydimethylsiloxane polymers, polyimine polymers, cryogels, aerogels or suitable organic polymers, for example imide-based compounds.

In a further embodiment of the photobioreactor 10 the micro-molded gas channels 24 and reaction channels 26 form the chip channel network structure that is positioned on a glass substrate 12 via cryo-printing.

In a further embodiment of the photobioreactor 10 concentrations and formation rates of oxygen and hydrogen are semi-continuously measured at the reactor outlet 16 by chip-coupled chromatographic measurements.

In a preferred embodiment biofilm capillary reactor 10 is a continuous microfluidic membrane photomicrobioreactor (μCMPB). Due to the low working volumes of the μCMPB system, safe operation—even under highly explosive oxyhydrogen conditions—is possible.

For the continuous microfluidic membrane photomicrobioreactor, the established 3D cryo-printing process is used to manufacture a customized chip design in minutes. The μCMPB is made of transparent, biocompatible, and gas-tight polymer, preferably an arcylate. The transparency of the chip material ensures that the reaction channel 26 containing the cyanobacteria is supplied with light. The 3D cryo-printing of the chip structures is carried out by depositing water droplet with a piezo printer on a chilled (−30 °C) glass substrate 12. The water immediately freezes and form the positive mold for the channel structure. The water structures are subsequently covered with chilled UV-curable acrylate and polymerized by UV light exposure. Thawing of the ice structures creates gas channels 24 and reaction 26 channels in the polymerized acylate. The oxygen-permeable membrane is created by acrylate-polydimethylsiloxane (PDMS) and poured between a trench between gas and reaction channel. The PDMS is subsequently polymerized and the whole chip is subsequently covered with acrylate to create a gas-tight chip. Hydrogels and aerogels can be printed as alternative oxygen-permeable membrane materials.

As illustrated in FIG. 2, the capillary serves as the reaction channel 26, with flanking capillaries as gas channels 24 designed to remove hydrogen and oxygen from the reaction channel 26. This is achieved by integrating different printed oxygen-and hydrogen-selective polymer membranes 22.

Thus, the reaction conditions in terms of oxygen sensitivity of the cell and the hydrogenase enzyme, the influence of the chemical equilibrium on the hydrogen formation reaction, as well as the mass transfer rates across the membrane can be specifically adapted to the formation rates of hydrogen and oxygen by modifying the membrane geometry, membrane exchange surface area, as well as the diffusivity of the membrane material. This information can be directly utilized to design membrane photobioreactor modules with integrated reaction and purification functionalities for the photohydrogen processes. Due to the possibility of controlling gas composition by flow rates in the μCMPB system, safe operation is possible by avoiding highly explosive oxyhydrogen conditions. Scale-up by numbering up theoretically allows safe operation even under highly explosive conditions due to very low working volumes. Concentrations and formation rates of oxygen and hydrogen are semi-continuously measured at the reactor outlet by chip-coupled chromatographic measurements.

By means of the biofilm capillary reactor according to the preset invention it is possible to have anaerobic conditions for up to two to three weeks combined with a continuous photohydrogen evolution. Longer process times and truly continuous processing without down times is likely possible in setups with a control of biofilm growth in the flow regime outside of the capillaries. This can be realized in scaled up variants of the biofilm reactor described here.

In summary, the invention makes it possible to produce continuously hydrogen from water by natural photosynthesis catalyzed by oxygen labile hydrogenases in the same microbial cell over weeks.

The present invention is further illustrated by the following examples, from which further features, embodiments, aspects and advantages of the present invention can be seen.

### EXAMPLES

### Example 1

### Hydrogen Formation With Suspended Cells (Batch)

To confirm the removal of biogenic oxygen and its impact on hydrogen evolution in a cyanobacterium, hydrogen and oxygen concentrations were measured by an established procedure using electrodes. For a duration up to 35 h anaerobiosis was maintained by the heterotrophic organism with either citrate or glucose as substrate for growing P. taiwanensis. This was accompanied by hydrogen production for several hours.

Hydrogen (H₂) evolution was detected during light and dark phases in vivo using Synechocystis PCC6803 M55 containing single or mixed species batch cultures measured by UniSense electrodes. To maintain anaerobic conditions also under photosynthetic conditions (light phase) cultures were supplemented with 10 mM glucose or citrate and either an enzyme mixture of 40 U glucose oxidase and 50 U catalase (God/Cat) or a defined amount of Pseudomonas cells. To maintain anaerobic conditions also under photosynthetic conditions, Synechocystis PCC6803 M55 cultures were supplemented with 10 mM glucose and either an enzyme mixture of 40 U glucose oxidase and 50 U catalase (God/Cat) or a defined amount of Pseudomonas cells. Pseudomonas taiwanensis cells did not produce hydrogen.

The application of Pseudomonas taiwanensis cells for depletion of oxygen by respiration of either citrate or glucose resulted in similar hydrogen evolution kinetics of Synecocystis sp. PCC6803 and its mutant M55 as the application of glucose and glucose oxidase as oxygen sinks. In addition, oxygen evolution by Synecocystis sp. PCC6803 was not impaired by respiration of Pseudomonas taiwanensis.

### Example 2

### Electrons for Production of Natural Photohydrogen can be Retrieved Directly From PSII

To identify the electron source of hydrogen evolved by the Synechocystis PCC6803 M55 mutant in mixed species cultivation with either glucose or citrate as carbon source, the herbicide DCMU (3-(3,4-dichlorophenyl)-1,1-dimethylurea) was added. DCMU is an herbicide and inhibits the electron transfer from PSII to the PQ-pool. In presence of DCMU only electrons originated from the fermentative carbon catabolismcan be used by the M55 mutant to evolve hydrogen. While without addition of the herbicide, both, electrons of the carbon catabolismand of the light driven water splitting of photosynthesis are involved in photohydrogen evolution. Hydrogen was determined directly via gas chromatography. Liquid co-cultures of the Synechocystis PCC 6803 M55 mutant and P. taiwanensis were transferred to a GC vail and the gas composition in the head space was analyzed at different time points. For an adequate gas exchange of the liquid and the gas phase, the samples were continuously mixed. To remove the already present oxygen in the vails by the heterotroph organism and to investigate the fermentative hydrogen evolution under dark conditions, the samples were incubated for one hour in darkness at the start of the measurement. Gas samples before and after 1 h of dark incubation were taken. Afterwards the mixed species cultivations were exposed to light, to induce photohydrogen evolution. Samples were taken after 3.5 h to investigate the start of photohydrogen evolution and after 20 h to gain insights in the amount of hydrogen evolving over a longer period.

The electron source of photohydrogen evolved by the M55 mutant (OD₇₅₀=3) was determined by co-cultivation in GC vials with P. taiwanensis (OD₄₅₀=3) in presence of either 10 mM glucose or 10 mM citrate and analyzed by head space gas chromatography. The GC vails were continuously mixed at 150 rpm to provide a gas exchange between gas and liquid phase, the incubation temperature was 30 °C. The GC was conducted with a ThermoScientific™ Trace 1310 Gas Chromatograph, carrier gas: argon, TG-Bond Misieve column (30 m length, 0.308 mm internal diameter, 0.3 μm film thickness), 50 °C operating temperature, 100 μl injection volume, detection by TCD (100 °C), mean and standard deviation of three independent biological experiments.

The measurements of the hydrogen production took place after Oh, 1h (dark incubation), 3.5 h and 20 h of co-cultivation (light conditions, 50 μE/m²). The amounts of hydrogen determined were up to 700 ppm after 20 hours for citrate as oxygen sink in Pseudomonas taiwanensis without addition of DCMU (no inhibition of PSII). With the same conditions, about 300 ppm hydrogen were formed when glucose was used instead of citrate for oxygen depletion (respiration) of Pseudomonas taiwanensis. In the same setup with addition of 20 μM DCMU to inhibit electron transfer of PSII to PQ-pool at the start of the experiment, about 300 ppm H₂ were detected in the headspace of the GC vail only in incubations with glucose. No hydrogen is detected under the same conditions with citrate instead of glucose for oxygen respiration by Pseudomonas taiwanensis. Hydrogen was produced in dark incubations with and without DCMU in these experiments only with glucose present. Only trace amounts of hydrogen were produced in experiments with DCMU present (presumably from intracellular storage compounds)

The hydrogen formation rate is higher under the conditions tested over time with citrate in light and not decreasing. Thus, no hydrogen (H₂) is formed from citrate in the dark, so no respiration driven hydrogen formation is running in the dark. In the dark, cells are respiring with glucose and are forming hydrogen from glucose. In contrast to mixed species cultivations with glucose, where hydrogen evolution of the Synechocystis PCC6803 M55 mutant seems to be driven mostly by electrons of the carbon catabolisma consistently higher amount of hydrogen was detected in the presence of citrate. An up to 2.7-fold higher amount of hydrogen was present in the headspace of the GC-vails at the end of the incubation. Moreover, the increased amount of hydrogen seems to originate mainly from the photosynthetic apparatus directly as no hydrogen (H₂) production could be observed when electron flow from PSII was inhibited by DCMU. This is in clear contrast to the use of glucose, which still allows hydrogen (H₂) production when electron flow from PSII is blocked.

### Example 3

### Exchange of Gas Phase

Co-cultivations of Synechocystis PCC 6803 M55 mutant and Pseudomonas taiwanensis were cultivated in gas tight containers with an equal volumetric gas phase. Different carbon sources were added to support oxygen (O₂) removal. Synechocystis PCC6803 M55 (OD₇₅₀=3) and Pseudomonas taiwanensis (OD₄₅₀=3) co-culture were supplemented with 10 mM citrate, gluconate or glycerol and hydrogen evolution in light was measured by GC. Cultures were kept under constant light. Periodically the hydrogen (H₂) concentration of the gas phase was measured by gas chromatography and afterwards gas phases were exchanged with fresh nitrogen (N₂) gas, for example every two days. Extracting hydrogen (H₂) from the culture showed effects upon activity of the hydrogenase and the long-term productivity for hydrogen. By simply exchanging gas phases, production phases in Synechocystis PCC6803 M 55/Pseudomonas cultures could be extended to a week, yet with a constant decreasing rate over the time period in this experimental setup.

### Example 4

### Continuous Production of Hydrogen for Weeks

The biofilm capillary photobioreactor system used is based on and using technology described in WO2018/162465 and WO2020/052762 which is a segmented flow biofilm capillary reactor system.

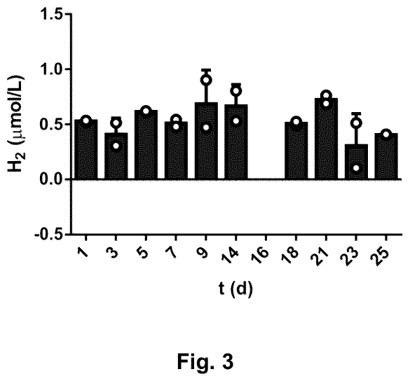

The biofilm capillary photobioreactors were inoculated with a co-culture of the Synechocystis PCC6803 M55 mutant (OD₇₅₀=1) and P. taiwanensis(OD₄₅₀=1). 0.4 g/L citrate were added to the biofilm medium as a carbon source for the heterotrophic organism to enable anaerobic conditions inside the biofilm capillary. As negative control, mixed species biofilms of the hydrogenase deficient Syn6803 Δhox mutant were cultivated under the same conditions. The biofilm cultivations and the hydrogen and oxygen measurements were performed under light conditions. A constant presence of hydrogen was detected in the outflow medium of the biofilm capillary for all days over three weeks of measurement. The results are given in FIG. 3.

FIG. 3 shows the H₂ production in mixed species biofilm capillaries (M55/Pseudomonas) .

Biofilms composed of Synechocystis PCC 6803 M55 and Pseudomonas taiwanensis were cultivated on the inner surface of a silicon tube (2 mm diameter, 200 mm length) and continuously supplied with nutrients by a constant stream (flow rate 52 μl/min) of sterile BG11 medium fed with 0.4 g/L citrate. Hydrogen measurements were started two weeks after biofilms were inoculated by using UniSense electrodes connected to the distal end of the capillary.

In FIG. 3 the bars are showing the mean ±SD of hydrogen concentration at the end of two individual capillaries containing Synechocystis PCC 6803 M55 and Pseudomonas taiwanensis at different time points after inoculation. Hydrogen analysis was not possible on day 16 due to analytical/technical problems. No hydrogen was produced in capillaries containing the Δhox mutant (control experiment).

These results demonstrate the feasibility of long term hydrogen formation in the light by oxygen sensitive hydrogenases in Synechocystis sp. PCC6803 cells with active PSII producing oxygen at high rates, provided that oxygen is efficiently depleted from the cytoplasm of these cells and the reaction vessel by means other than respiration of the phototrophic cell.

### Example 5

### Separation of Gas Phase

The experimentally verified photobioreactor according to the present invention enables the complete removal of photosynthetic oxygen from the microcapillary reaction space at a cell concentration of 7 gCDW/L (which corresponds to an OD₇₅₀=40) at a photon flux density of 50 μE m⁻¹ s⁻¹ (photobioreactor concept see FIG. 2). The photobioreactor uses the passive, concentration-driven removal of biogenic oxygen from the reaction channel. The photobioreactor consists of two parallel, meandering channels for the cyanobacteria suspension or biofilm. The two channels are separated by an oxygen and hydrogen permeable membrane made of PDMS, aerogels or cryogels. The gas channel is continuously flushed with nitrogen to enable the passive transfer oxygen from the reaction channel into the gas channel. The high membrane surface-to-reaction volume makes the passive oxygen removal efficient in comparison to larger scale reactor concepts.

This demonstrates, that the microcapillary-membrane photobioreactor (μCMPB) concept is a powerful tool to design strategies for solving the oxygen issue in white hydrogen production processes. The high efficiency of the oxygen removal in combination with the high area-specific productivity of microreactors shall stimulate the development of scalable μCMPB concepts towards its application as printable low-cost production-scale photomicrobioreactor modules for scalable decentralized white hydrogen processes.

### Example 6

### Generation of M55 Like Mutants of Synechocystis sp PCC6803 by Reengineering

Starting from Synechocystis PCC6803, a new mutant thereof, the ndhB knockout strain, was constructed by inserting a Kanamycin resistance gene cartridge in the ndhB gene as it was done by Teruo Ogawa before. After several rounds of cultivation, 5 out of 6 strains were shown to be fully segregated, while ndhB6 resulted in a mix phenotype showing both wild type, as well as an ndhB knockout genotype, as proven by col-PCR (expected size: WT 2842bp, ndhB knockout 3637bp). Synechocystis sp PCC6803 M55 and the newly constructed ΔndhB variants were compared for anaerobic hydrogen (H₂) production under illumination. The tests were done in 3 ml BG11 medium after adding of 10 mM Glucose, 40 U glucose-oxidase and 50 U catalase measured with an H₂-micro-respiration sensor from UniSense.

ΔndhB fully segregated strains exhibited higher production of hydrogen (H₂) over a longer time period with a small peak (roughly 10 min at 30 mol/L H₂), while the M55 mutant maintained lower level of hydrogen (H₂) formation over a longer time period (roughly one hour at 18 μmol/L).

These experiments demonstrate the possibility to reengineer the photo hydrogen forming phenotype of Synechocystis sp. PCC6803 M55.

1. A method for producing hydrogen from water, the method comprising:
co-cultivation of hydrogen(H2)-releasing cyanobacteria with heterotrophic, oxygen respiring bacteria and an organic electron source under anaerobic conditions in a reaction medium containing water and/or a biofilm; and
exposure to light to induce photohydrogen evolution,
wherein the oxygen is exclusive extracellular removed with a membrane area-specific withdrawal rate of 0.5 to 9 μmolO₂*mm²*h⁻¹ to an oxygen concentration of 0 to 12 % v/v.

2. The method according to claim 1, wherein the organic electron source is naturally metabolized by the heterotrophic, oxygen respiring bacteria and not metabolized by hydrogen-releasing cyanobacteria.

3. The method according to claim, wherein the organic electron source is selected from citrate, gluconate or glycerol.

4. The method according to claim 1, wherein the organic electron source is citrate.

5. The method according to claim 1, wherein the hydrogen-releasing cyanobacteria are selected from the genus Synechocystis.

6. The method according to claim 1, wherein the hydrogen-releasing cyanobacteria are the genetically modified cyanobacterial strain Synechocystis sp. PCC6803 M55 or any derivative thereof.

7. The method according to claim 1, wherein the hydrogen-releasing cyanobacteria are genetically and phenotypically different strains of Synechocystis PCC6803 obtained by molecular breeding.

8. The method according to claim 1, wherein the heterotrophic, oxygen respiring bacteria are selected from the genus Pseudomonas, particularly Pseudomonas sp. VLB120.

9. The method according to claim 1, wherein the oxygen is physically removed from the gas phase with a membrane.

10. The method according to claim 1, wherein the gas phase above the reaction medium is periodically exchanged, by nitrogen (N₂) gas.

11. A photobioreactor configured to perform the method according to claim 1, the photobioreactor comprising a chip made of a transparent, biocompatible, and gas-tight polymer, capillary members which are arranged meander-shaped, parallel on the chip, wherein at least one capillary member is the reaction channel flanked by at least one capillary member which is the gas channel and between the reaction channel and the gas channel are integrated oxygen- and hydrogen-selective polymer membranes to remove hydrogen and oxygen from the reaction channel.

12. The photobioreactor according to claim 11, wherein the gas-tight polymer is an acrylate.

13. The photobioreactor according to claim 11, wherein the membranes are made of polydimethylsiloxane polymers, polyimine polymers, cryogels or aerogels.

14. A method comprising:
producing, via a photobioreactor, 12 to produce hydrogen with the method according to claim 1claims 1 to 10.
